(19) United States
(12) Patent Application Publication
(10) Pub. No.: US 2004/0214243 A1
(43) Pub. Date: Oct. 28, 2004

(54) Differential determination of hemoglobins

(75) Inventors: Alexander Burshteyn, Pembroke Pines, FL (US); Andreas Van Agthoven, Marseille (FR); Frank J. Lucas, Boca Raton, FL (US); Enrique Rabellino, Miami, FL (US)

Correspondence Address: BECKMAN COULTER, INC., P.O. BOX 169015, MAIL CODE 32-A02, MIAMI, FL, 33116-9015, US

(73) Assignee: Beckman Coulter, Inc., Fullerton, CA (US)

(21) Appl. No.: 10/423,544

(22) Filed: Apr. 25, 2003

(51) Int. Cl. G01N 33/567; G01N 33/53; G01N 33/555

(52) U.S. Cl. 435/7.21; 436/520

(57) ABSTRACT

The present invention relates to reagents for analyzing hemoglobin in a sample using a pan-hemoglobin antibody conjugated to a detectable marker and one or more affinity reagents that are conjugated to a detectable marker that specifically bind to hemoglobin types and/or variants. The present invention is further drawn to flow cytometric methods using the reagents.

### FIELD OF THE INVENTION

The present invention relates to reagents for analyzing a hemoglobin type or variant. In addition, the present invention relates to a flow cytometric method using the reagents.

### BACKGROUND OF THE INVENTION

Normal adult hemoglobin A (Hb A) consists of two α (alpha) and two β (beta) chains (α₂β₂). A second normal adult hemoglobin A₂ (Hb A₂) consists of two α and two δ (delta) chains (α₂δ₂). The blood of normal adult humans contains Hb A as the major hemoglobin species and Hb A₂ as a minor hemoglobin species. Human fetuses and newborn infants produce mainly fetal hemoglobin F (Hb F) which consists of two a chains and two γ (gamma) chains. Additionally, the θ (theta) chain, the ζ (zeta) chain and the ε (epsilon) chain have been observed in early human embryos.

One of the first abnormal hemoglobin discovered was hemoglobin S (Hb S) which is responsible for sickle cell anemia. Hb S is the result of a substitution of a valine residue for the glutamate residue normally found at position 6 of the β chain. Another relatively common abnormal hemoglobin is hemoglobin C (Hb C).

In addition, approximately 90% of total hemoglobin is nonglycosylated. The major fraction of nonglycosylated hemoglobin is nonglycosylated Hb A, referred to as Hb A₀. Glycated hemoglobin (GHb) refers to a series of minor hemoglobin components that are formed via the attachment of various sugars to the hemoglobin molecule. The human erythrocyte is freely permeable to glucose. Within each erythrocyte, GHb is formed at a rate that is directly proportional to the ambient glucose concentration. The reaction of glucose with hemoglobin is nonenzymatic, irreversible and slow, so that only a fraction of the total hemoglobin is glycated during the life span of an erythrocyte (120 days). As a result, the measurement of GHb provides a weighted “moving” average of blood glucose levels that can be used to monitor long-term blood glucose levels, providing an accurate index of the mean blood glucose concentration over the preceding 2 to 3 months. The most important clinical application of this is in the assessment of glycemic control in a diabetic patient.

Hemoglobin A_{1C} (HbA_{1C}) is one specific type of glycated hemoglobin and is the most important hemoglobin species with respect to diabetes. HbA_{1C} arises by reaction of a terminal valine amine group in the β chain with the aldehyde group of glucose to give an unstable aldimine. Rearrangement of the aldimine gives HbA_{1C}, which is characterized by a β-ketoglycoside linked to the valine amine group. The total amount of hemoglobin that is HbA_{1c} is approximately 3 to 6% in nondiabetics, and 20% or greater in diabetes that is poorly controlled. Goldstein, et al., Clin. Chem. 32: B64-B70 (1986). The Diabetes Control and Complications Trial (DCCT) Research Group reported that a 1% change in GHb (% HbA_{1C}) represents an average change of 300 mg/L in blood glucose levels over the preceding 120 days. Thus, the determination of the concentration of HbA_{1C} is useful in diagnosing and monitoring diabetes mellitus.

Numerous procedures have been used to identify and characterize hemoglobins. Traditionally, these methods have included electrophoresis, isoelectric focusing, HPLC and macro-chromatography. In addition, flow cytometry has been used to analyze particular hemoglobin types and/or variants.

Flow cytometry provides a rapid and efficient method for the analysis of blood samples in which single red blood cells are analyzed. When flow cytometry has been used to analyze specific hemoglobin types and/or variants, a monoclonal antibody specific to a particular hemoglobin of interest has been used to measure the population of the specific hemoglobin types and/or variant and the total hemoglobin population has been determined by using either light scatter to identify the total red blood cell population or by using or a monoclonal antibody specific for Hb A. Dover, et al. Blood 61:4 1109-1113 (1987); Jensen et al. Hemoglobin 9 (4) 349-362 (1985). As a third method, the total hemoglobin is determined by identifying the total red blood cell population by labeling the glycophorin A on the red blood cells.

However, none of these methods is capable of accurately determining the total hemoglobin population. Using light scatter to identify the red blood cell population based on size results in an erroneously high measurement for the total hemoglobin population because of non-red blood cell particulates that give false positives in the light scatter window. In addition, basing the total hemoglobin on the glycophorin A labeling will result in an artificially high value because all cells of red blood cell lineage, i.e. nucleated red blood cells, reticulocytes and mature red blood cells, express glycophorin A protein but not all of cells of red blood cell lineage contain hemoglobin. Nucleated red blood cells and reticulocytes can have only trace or small amounts of hemoglobin. Using an antibody to Hb A results in an erroneously low number for total hemoglobin because only 90-95% of the hemoglobin in a normal subject is in the A form and there can be even less in an abnormal patient.

An additional limitation with using flow cytometry to analyze hemoglobin results from the lack of color compensation reagents required for an accurate measurement. When more than one fluorescent reagent is used in a flow cytometric analysis of a single sample (for example, fluorescein and rhodamine), the overlap in the fluorescent spectra of the reagents results in an inaccurate measurement of the respective populations, due to bleed over fluorescence from one fluorescent spectra to the other. To compensate for errors in analysis caused by the spectral overlap, reagents are needed which allow the instrumentation to be set to eliminate the artificial positive signal caused by the bleed over fluorescence. To establish the color compensation on a flow cytometer, a set of relevant fluorochrome reagents that discretely bind to cells is needed. In the subtraction compensation method, each reagent is coupled to a different fluorophore and spectral overlap is subtracted. In the full matrix compensation method, one reagent coupled to each different fluorophore used is needed. Bagwell, C B et al. Ann N Y Acad Sci.20:677 167-84 (1993). However, no color compensation reagent system currently exists for red blood cells. As a result, multicolor flow cytometric analysis of red blood cells can be inaccurate.

For these reasons, it has not been possible to use flow cytometry to obtain an accurate measurement of hemoglobin by immunofluorescence. As discussed above, given that a 1% change in a particular hemoglobin population can be indicative of a pathological state, an accurate sensitive method is needed for the rapid analysis of hemoglobin in a sample, using flow cytometry.

The present invention overcomes these drawbacks and provides an accurate method of using flow cytometry to analyze hemoglobin in a sample. The present invention further provides a color compensation system to enable the accurate measurements using multicolor flow cytometry analysis of red blood cells and red blood cell components, such as hemoglobin types and/or variants.

### SUMMARY OF THE INVENTION

The present invention concerns a method of analyzing one or more hemoglobin types and/or variants in a sample comprising mixing a test sample from a patient with a pan-hemoglobin antibody that is conjugated to a first label and a hemoglobin type or variant-specific affinity reagent that is conjugated to a second label; measuring the test sample to determine a signal generated from the first label on the pan-hemoglobin antibody and a signal generated from the second label on the hemoglobin type or variant-specific affinity reagent; comparing the signal from said pan-hemoglobin antibody and said hemoglobin type or variant specific affinity reagent; and reporting the result of the comparison.

The present invention further encompasses a conjugated antibody product comprising a pan-hemoglobin antibody conjugated to a detectable label. An additional aspect of the invention relates to a conjugated antibody product that can be used as a control product. In addition, the control product can contain a known quantity of one or more hemoglobin types and/or variants. Another aspect of the present invention encompasses the conjugated antibody product further comprising one or more antibodies to white blood cells and white blood cell components for a whole blood assay.

In a further embodiment of the present invention, the conjugated antibody product can comprise a plurality of pan-hemoglobin antibodies each conjugated to different fluorescent labels. In addition, the color compensation kit can comprise a pan-hemoglobin antibody conjugated to a detectable label and at least one additional hemoglobin type or variant-specific affinity reagent that is conjugated to another detectable label wherein the antibody and each additional hemoglobin type or variant specific affinity reagent has a detectable label is different from the other. An example of such embodiment comprises a pan-hemoglobin antibody conjugated to a first detectable label and an antibody that binds specifically to glycophorin A having a second detectable label.

The present invention also encompasses diagnostic and prognostic methods for diabetes mellitus which comprise reacting a patient sample with an antibody to Hb A_{1c}, wherein said antibody is conjugated to a first detectable label and a pan-hemoglobin antibody that is conjugated to a second detectable label; measuring the test sample to determine a signal generated from the first label on the pan-hemoglobin antibody and a signal generated from the second label on the Hb A_{1c} specific affinity reagent; and comparing the signal from said pan-hemoglobin antibody and said Hb A_{1c} specific affinity reagent.

An additional aspect of the invention is drawn to a method for monitoring treatment compliance of a patient with diabetes mellitus, which comprises reacting a patient sample with an antibody to Hb A_{1c} and/or glycosylated hemoglobin, wherein in said antibody is conjugated to a first detectable label, and a pan-hemoglobin antibody that is conjugated to a second detectable label; measuring the test sample to determine a signal generated from the first label on the pan-hemoglobin antibody and a signal generated from the second label on the antibody to Hb A_{1c} and/or glycosylated hemoglobin; and comparing the signal from said pan-hemoglobin antibody and said Hb A_{1c} and/or glycosylated hemoglobin antibody to a reference value.

### BRIEF DESCRIPTION OF THE DRAWINGS

FIGS. 1A-3C depict dot plots and histograms from a dual color flow cytometry analysis of a red blood cell sample labeled with pan hemoglobin antibody and anti-HbA_{1C} antibody.

FIG. 1A-C comprises one scatterplot and two histograms which relates to control products.

FIG. 2A-C comprises one scatterplot and two histograms which relate to using the reagents of the present invention for color compensation.

FIG. 3A-C comprises one scatterplot and two histograms which relate to red blood cells from a non-compliant diabetic patient (type 1) stained with Pan-Hb-FITC (FL1) and anti HbA_{1C}-PE (FL2) fluorescent reagents.

FIG. 4A-C comprises three scatterplots that relate to flow cytometry analysis of a cell preparation (Immuno-Trol™ flow cytometry control product, Beckman Coulter, Inc., Fullerton, Calif.) containing RBCs from a Sickle cell anemia patient labeled with Pan-hemoglobin antibody and anti-S hemoglobin antibody.

FIGS. 5A-7B are dot plots and histograms of a dual color flow cytometry analysis of two cell preparations. The first cell preparation is Immuno-Trol™ control product (Beckman Coulter, Inc.) spiked with cord blood red blood cells and labeled with Pan hemoglobin antibody and labeled with an antibody that binds to the i antigen expressed by embryonic red blood cell (anti i antigen antibody) and the second cell preparation is normal red blood cells spiked with cord blood red blood cells and labeled with Pan hemoglobin antibody and labeled with anti i antigen antibody.

FIG. 5A-C comprises one scatterplot and two histograms which relate to a control product.

FIG. 6A-B comprises two histograms which relate to color compensation using single color histograms for color compensation showing the distribution of Immuno-Trol™ cells stained with 1.1 μg Pan-Hb-FITC monoclonal antibody (FL1) and 0.1 μg Glycophorin A-PE monoclonal antibody (FL2).

FIG. 7A-B comprises one scatterplot and one histogram that relate to a test assay of hemoglobins.

FIGS. 8A-9C are scatter plots and histograms of a dual color flow cytometry analysis of a normal blood sample spiked with cord blood red blood cells labeled with pan hemoglobin antibody and anti i antigen antibody.

FIG. 8A-C comprises one scatterplot and two histograms that relate to control products.

FIG. 9A-C comprises three histograms that relate color compensation and test assay.

### DETAILED DESCRIPTION OF THE INVENTION

The present invention provides an accurate quantitative method to analyze hemoglobin in a sample. The preferred method uses flow cytometry to analyze individual cell that passes through the measurement zone. As appreciated by those skilled in the art, the present invention can also be practiced on a fluorescent microscope, but the time to analyze a sample will be substantially increased. The present invention further provides a color compensation reagent system to enable to the use of multicolor flow cytometry for an accurate analysis of red blood cells and red blood cell components, such as hemoglobin types and/or variants.

“Pan-hemoglobin” antibody is an antibody that binds to a common antigenic determinant on the hemoglobin chains resulting in the labeling of the total hemoglobin population. For example, pan-hemoglobin antibody will bind at least to the a hemoglobin chain which is common to all hemoglobin types and variants. Moreover, the pan-hemoglobin antibody can also bind to the α (alpha) and δ (delta) hemoglobin chains which will also result in the labeling of the total hemoglobin population. Preferably the pan-hemoglobin antibody is a monoclonal antibody. However, the present invention also contemplates that a pan-polyclonal antibody could be used providing that it results in the labeling of the total hemoglobin population. Various pan-hemoglobin antibodies are commercially available in an unconjugated form. Examples of pan-hemoglobin antibodies can be obtained from the following manufacturers: a) monoclonal antibodies are available from Cortex Biochem, Inc., San Leandro, Calif., Product ID CR8001M, Name: Hemoglobin (Alpha Chain) Description: Anti-hemoglobin (alpha); Biodesign International, Kennebunk, Me., Catalog No. H67696M, Name: Human Hemoglobin alpha chain, Description: Monoclonal anti-hemoglobin (alpha chain); Fitzgerald International, Inc., Concord, Mass., Catalog: 10-H03, Name: Hemoglobin whole molecule (human); and b) polyclonal antibodies are available from Accurate Antibodies, Westbury, N.Y., Product ID: IMS-02-068-02, Name: Hemoglobin Chicken Anti Human; and Product ID: BMD-J16, Name: Hemoglobin Goat Anti Human; and Product ID: BYA-1006-1, Name: Hemoglobin Rabbit Anti Human. However, pan-hemoglobin antibodies have not been used in flow cytometry or been conjugated to a detectable label. In addition, several preconjugated hemoglobin antibodies specific to a particular hemoglobin type and/or variant are available from a variety of commercial sources.

Prior to the date of this invention, a monoclonal antibody specific for hemoglobin A had been used to measure the total hemoglobin in a sample. Campbell, et al. Cytometry 35:242-248 (1999). However, unlike pan-hemoglobin antibodies, monoclonal antibodies to hemoglobin A only react with 90-95% of the normal total hemoglobin population. The present inventors have for the first time conjugated pan-hemoglobin antibody to a detectable label. Thus, one aspect of the present invention is drawn to a pan-hemoglobin antibody conjugated to a detectable label that is suitable for use as a flow cytometry reagent.

The use of a pan-hemoglobin antibody for the detection of total hemoglobin in a sample when used in combination with an affinity reagent, such as an antibody that is specific for a distinct hemoglobin type and/or variant, results in an accurate method, preferably by flow cytometry, for analyzing the amount of type and/or variant present in the sample. Hemoglobin types include, but are not limited to, HbA_{1C}, HbA, HbA₂, embryonic Hb, HbS, HbF, HbC, HbD, HbE and glycosylated Hb. In addition, hemoglobin variants include many hemoglobin derivatives of the hemoglobin types. Hemoglobin variants often arise as the result of a single mutation in the amino acid sequence of a hemoglobin type. Using the present invention, any hemoglobin can be detected, preferably by flow cytometry, for which there is a specific affinity reagent, such as an antibody, that can be conjugated to a detectable label. Moreover, antibodies that bind to intracellular molecules or antigens and bind to surface membrane molecules or antigens can be combined with the pan-Hb to provide additional information about the cell.

The detectable label on the pan-hemoglobin antibody and hemoglobin type and/or variant affinity reagent can be any label that is detectable, preferably using flow cytometry, such as a fluorophore. Fluorophores include both fluorescent labels that exist in a fluorescent state and fluorochromes that fluoresce upon excitation. Numerous fluorophores suitable for the present invention are commercially available from several companies, such as those available through Molecular Probes, Inc., Eugene, OR.

Examples of suitable fluorophores include, but are not limited to the Alexa Fluor dye series, including Alexa 350, Alexa 430, Alexa 488, Alexa 532, Alexa 546, Alexa 555, Alexa 568, Alexa 594, Alexa 633, Alexa 647, Alexa 660, Alexa 700 and Alexa 750, BODIPY dyes, fluorescein, Oregon green, rhodamine green, tetramethylrhodamine, lissamine rhodamine B, rhodamine Red-X, A-rhodamine, X-rhodamine, Texas Red, Texas Red-X, naphthofluoroscein, LaserPro IR 790, carboxyrhodamine 6G, QSY dyes, NANOGOLD sulfosuccinimidyl ester, Cascade Blue, coumarin derivatives, naphthalenes, pyrenes, pyridyloxazole derivatives, Cascade Yellow, Dapoxyl dye, Eosin derivatives, pyridyloxazole derivatives, benzoxadiazole derivatives, Lucifer Yellow, AMCA, Marina Blue, Pacific Blue, phycoerythrin (PE), PE based tandem fluorochromes (PE-Tx Red, PE-Cy5, PE-Cy5.5, PE-Cy7), Cy3, Cy3.5, Allophycocyanin (APC), APC based tandem fluorochromes (APCCy7, APCCy5.5, Cy5.5, Cy7), and chromatographic maleimides.

Conjugation of the fluorophore to the antibodies used in the present invention can be done using conventional and well-known techniques. The conjugated Pan-Hgb product can be packaged and sold as a lyophilized product or in as a liquid product. The lyophilized product will tend to have greater shelf storage than the liquid product. The liquid product will contain an appropriate buffer, such as phosphate buffer solution (PBS) and at least one preservative.

The method of the present invention can be used for analyzing hemoglobin in patient samples. Using the method of the present invention, the percentage of red blood cells that contain a particular type and/or variant of hemoglobin can be determined. More specifically, using the Pan-Hb conjugate enables the determination of a number of red blood cells and the type and/or variant Hb conjugate enables the determination of the number of red blood cells that contain the type and/or variant such that a percentage of red blood cells that contain the type/variant Hb can be determined. In another embodiment, the percent concentration of a particular hemoglobin type and/or variant to the total hemoglobin content can be determined by first measuring the total concentration of hemoglobin in a sample using the signal intensity from the labeled pan-hemoglobin antibody and a reference standard of a known amount of hemoglobin and then determining the concentration of the type and/or variant by using the signal intensity from the labeled type and/or variant hemoglobin antibody and the reference standard of the known amount of type and/or variant hemoglobin. In a further embodiment, the percent concentration of a particular hemoglobin type and/or variant to the total hemoglobin content can be determined by first measuring the total concentration of hemoglobin in a sample using other suitable means, such as absorbance or light scatter, and the concentration of the type and/or variant can be determined by a correlation table of the mean concentration of the type and/or variant contained in the cells identified by the labeled type and/or variant hemoglobin antibody. U.S. Pat. No. 5,686,309 is hereby incorporated by reference in its entirety. Still further, it is also within the contemplation of the present application to use the present invention to determine the mean number of red blood cells per blood volume containing a particular hemoglobin type and/or variant, such as hemoglobin A_{1C} and/or glycosylated hemoglobin, in a test sample.

The present invention can have both diagnostic and prognostic applications. For diagnostic applications, the presence and/or amount of particular hemoglobin types and/or variants can diagnose the presence and extent of several pathological conditions. More specifically, the present invention can provide valuable diagnostic and prognostic information related to hemoglobinopathies and diabetes mellitus.

Hemoglobinopathies represent an heterogeneous group of disorders characterized for the presence of hemoglobin types and/or variant other than Hb A₀ and Hb F. For example, Hb A₂ has been associated with some forms of β-thalassemias, HbA_{1C} and glycosylated Hb have been associated with diabetes and collagen disorders. Hb S has been associated with sickle cell disease. Hb C and Hb D have been associated with Hb C and Hb D diseases, respectively and Hb E has been associated with Hb E disease and β-thalassemia. Furthermore, while Hb F is expressed in normal fetuses and newborns it is also been associated with sickle cell anemia.

Of particular importance is the hemoglobin Hb A_{1c}. The total amount of hemoglobin that is Hb_{A1c} is approximately 3 to 6% in nondiabetics, and 20% or greater in diabetes that is poorly controlled (Goldstein, DE, et al., Clin. Chem. 32: B64-B70 (1986)). The Diabetes Control and Complications Trial (DCCT) Research Group reported that a 1% change in GHb (% HbA_{1C}) represents an average change of 300 mg/L in blood glucose levels over the preceding 120 days. In addition, it has been suggested that hemoglobin A_{1c} monitoring can be used as a pre-diabetes screen because patients can show elevated levels of hemoglobin A_{1c} before they have an abnormal glucose tolerance screen. Thus, the determination of the concentration of hemoglobin A_{1c} in a patient test sample is useful both in diagnosing diabetes mellitus and in monitoring the treatment of the disease.

Recently issued Guidelines that have been approved by the Professional Practice Committee of the American Diabetes Association, recommend that hemoglobin A_{1C} and/or glycosylated hemoglobin should be routinely measured in all patients with diabetes mellitus to monitor their glycemic control and compliance with treatment regimes. Sacks, et al., Clin. Chem. 48:436-472 (2002). At the same time the Guidelines noted the inconsistencies and inaccuracies of different laboratory assays currently being used to monitor hemoglobin A_{1c} and glycosylated hemoglobin.

The present method provides an accurate and consistent means of analyzing the hemoglobin A_{1c} and glycosylated hemoglobin in a patient sample. Using the method of the present invention the percent of cells containing hemoglobin A_{1c} and/or glycosylated hemoglobin can be determined by measuring the total number of cells containing hemoglobin using the signal from the labeled pan-hemoglobin antibody and measuring the total number of cells containing hemoglobin A_{1c} and/or glycosylated hemoglobin using the signal from the labeled antibodies specific for these hemoglobins. Alternatively, the concentration of hemoglobin A_{1c} and/or glycosylated hemoglobin can be determined by measuring the total concentration of hemoglobin in a sample using the signal from the labeled pan-hemoglobin antibody and a reference standard of a known amount of hemoglobin and then measuring the total concentration of hemoglobin A_{1c} and/or glycosylated hemoglobin in the sample using the signal from the labeled hemoglobin A_{1c} and/or glycosylated hemoglobin and a reference standard for a known amount of the hemoglobin A_{1c} and/or glycosylated hemoglobin.

The present invention is also drawn to a method of screening for diabetes mellitus by reacting a patient test sample with an antibody to hemoglobin A_{1c} that is conjugated to a detectable label and with a pan-hemoglobin antibody that is conjugated to a second detectable label. Preferably the method utilizes a flow cytometer. The amount of hemoglobin A_{1c} present in the sample can be determined and the results compared to a reference of hemoglobin A_{1c} that is found in a comparable normal patient population.

In addition to screening applications, the present method also has prognostic applications. For example, even a small change (up to 1%) in the hemoglobin A_{1C} levels of a diabetic patient has been correlated to a 35% increase of long term complications associated with diabetes. The method of the invention has the sensitivity to accurately measure such minor changes in the levels of hemoglobin types and/or variants.

In addition, the present invention can be used as a rapid and efficient means of monitoring treatment compliance in diabetes patients by using the reagents and methods previous described herein.

The present invention is further drawn to a control product containing at least one conjugated pan-hemoglobin antibody conjugated to a detectable label and a known quantity of one or more hemoglobin types and/or variants. For example, the control product can contain a labeled pan-hemoglobin antibody and a known quantity of one or more hemoglobin type or variant, such as Hb A₀, Hb F, and Hb S. The control product of the invention can be used for a flow cytometer. The control product can also contain specific affinity reagents, such as antibodies, to one or more hemoglobin types and/or variants, which have been conjugated to detectable labels, such as FITC, PE, PE-Tex Red or PE-Cy5.

The components of the control product can be packaged as a single unit. Within the unit packaging, the individual reagents, such as the conjugated pan-hemoglobin antibody; the known type or known quantity of a hemoglobin type and/or variant affinity reagent can be contained in separate containers, such as vials, or can be premixed together. The reagents in the control product can be provided in a reconstituted form or can be lyophilized for appropriate reconstitution by the end user. The packaged control products can also contain appropriate instructions for use and storage of the reagents.

An additional aspect of the invention provides color compensation kit containing reagents and a method of establishing the color compensation for multicolor analysis of red blood cell analysis on a flow cytometer. When more than one fluorescent reagent is used in the same sample analysis in a flow cytometry analysis (for example, fluoroscein and PE) the overlap of the fluorescent spectra of the reagents can result in an inaccurate measurement of the respective populations because of the bleed over of the fluorescence signal from one fluorescent spectra into the other fluorescent spectra from the sample analysis. To compensate for errors in analysis caused by the spectral overlap, reagents are used which allow the instrumentation to be set to eliminate the artificial positive signal caused by the bleed over fluorescence signal.

To establish the color compensation on a flow cytometer, two or more reagents are needed that discretely bind to the same cell population. Each reagent is coupled to a different fluorophore. With the color compensation reagents of the present invention, glycophorin A is labeled with a first fluorophore. Glycophorin A is a sialoglycoprotein that is specific for red blood cell linage cells and present on human erythroid precursor cells through mature red blood cells. In addition, the red blood cells are labeled with the pan-hemoglobin antibody conjugated to a second fluorophore. Thus, with the color compensation system of the present invention, red blood cells are labeled with two discrete red blood cell specific labels, i.e. an antibody that binds specifically to glycophorin A and pan hemoglobin antibody. Using these reagents, accurate multicolor flow cytometry analysis can be done with red blood cells.

Most flow cytometers today are capable of a multi color system of analysis. In addition, instrumentation and software are available beyond five-color analysis. Color compensation in these systems can be achieved by full matrix compensation using reagents conjugated separately to the different fluorochromes to be used. A software program then establishes color compensation for each fluorochrome. With the color compensation system of the present invention, a monoclonal antibody to glycophorin A is conjugated to three different fluorescent labels, for example, FITC, PE, and PE-Tx Red. In addition, pan hemoglobin antibody is separately conjugated to three different fluorescent labels, for example, PE, PE-Tx Red and PE-Cy5. Red blood cells are then labeled with the selected conjugated antibodies. For example, to obtain matrix color compensation, red blood cells labeled with the following antibody conjugates can be prepared as follows:

1) Glycophorin A-FITC+pan Hb-PE

2) Glycophorin A-PE+pan Hb-PE-Tx Red

3) Glycophorin A-PE+pan Hb-PE-Cy5

4) Glycophorin A-PE-Tx Red+pan Hb-PE-Cy5

Four samples of red blood cells each containing a different labeled pair of Glycophorin A antibody and pan hemoglobin antibody are run through a flow cytometer and the color compensation can be determined. The color compensation system for red blood cells can be adapted for use with a five-color analysis or greater.

The present invention also contains color compensation kits for multicolor analysis of red blood cells using flow cytometry. With the color compensation kits the color compensation reagents described above will be packaged as a unit. Within the unit, the glycophorin A labels and the pan-hemoglobin antibodies can be contained in the same or separate vials. The reagents in the kit can be provided in a reconstituted form or can be lyophilized for appropriate reconstitution by the end user. Also within the kit can be appropriate instructions and/or software regarding the use and storage of the color compensation reagents.

### EXEMPLIFIED EMBODIMENTS OF THE INVENTION

### Example 1

### RBC Preparation

A. Crosslinking of RBC-200 μL of a blood sample was pipetted into a test tube. To the blood sample 3.0 ml of twice diluted Reagent #1 was added, the sample was vortexed for 5 sec. and mixed on a roller mixer for 35 min. for whole blood. The sample was then centrifuged for 5 min. at 200 g, 1100 rpm and the supernatant removed.

B. Permeabilization of crosslinked RBC-Red blood cells can be permeabilized using known techniques and reagents, such as those disclosed in U.S. Pat. No. 6,534,279 to Van Agthoven, et. al., the entire contents of which are hereby incorporated by reference. After centrifuging and removing the supernatant, the pellet was resuspended with 3.0 ml of ten times diluted Reagent #2, sonicated to disperse for 10 sec., vortexed and mixed on a roller mixer for 5 min. The sample was then centrifuged for 5 min. at 200 g, 1100 rpm and the supernatant removed. At this point the sample can be stored for up to two weeks refrigerated or the pellet resuspended in 0.5 ml of PBS.

C. Blocking, stabilization and storage—If the sample is to be stored, the pellet can be resuspended in 3.0 ml of ten times diluted Reagent #3, vortexed 5 sec. and mixed on a roller mixer for at least 1 hour (up to 3 hours). The sample can then be stored refrigerated up to 2 weeks. After storage, the sample is washed two times on a centrifuge with 3 ml of PBS each time and centrifuged for 10 min., 200 g, 1100 rpm. After centrifuging, the supernatant is removed and the pellet resuspended to 0.5 ml with PBS for antibody binding.

Reagent #1 (500 ml)

37% formaldehyde solution, 270 ml

500,000 MW dextran sulfate, 0.5 g

20×PBS, 25 ml

D(+) Trehalose, 150 g

Distilled water to 500 ml

pH to 5.5 with HCL

Reagent #2 (500 ml)

Citric acid, 10.5 g

10% SDS solution 15.5 ml

D(+) Trehalose, 150 g

Distilled water to 500 ml

Reagent #3 (500 ml)

Tween 20, 100 ml

D(+) Trehalose, 100 g

Trizma base, 3.03 g

NaCl 2.90 g

Distilled water to 500 ml

pH to 7.4 with HCL

### Example 2

### Mouse lgG Isotype Controls and Color Compensation Controls

In this Example, three tubes were prepared as follows:

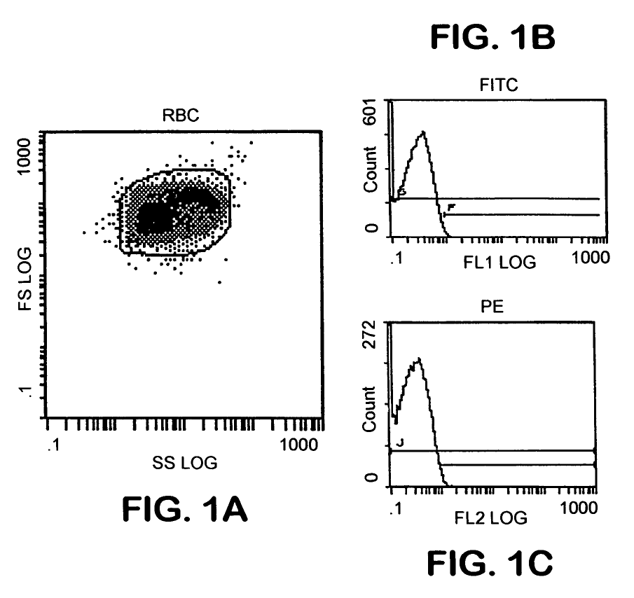

Tube 1: Mouse isotype control tube was prepared by incubating 20 μl of prepared RBC with 10 μl of mouse lgG1-FITC/ mouse lgG1-PE (1.1 μg: 1.1 μg). FIG. 1A-C comprises one scatterplot and two histograms which relates the control product. FIG. 1A is a dot plot showing the red cell distribution gated on forward versus side angle light scattering (log scale). FIG. 1B is a histogram that depicts the background fluorescence staining signal for FL1 as determined by an MslgG1-FITC isotype control (log scale). FIG. 1C is a histogram that depicts the background fluorescence signal for FL2 as determined by an MslgG1-PE isotype control (log scale). In both FIG. 1B and 1C, linear analysis regions are assigned in the histograms.

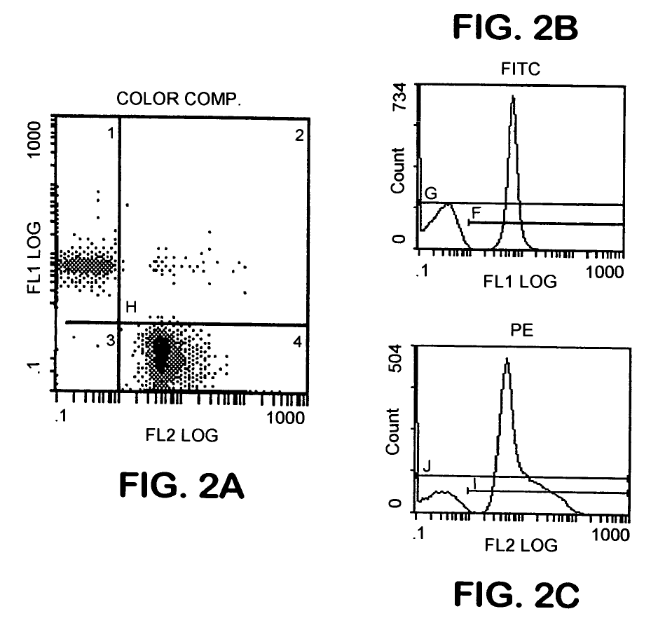

Tube 2 and Tube 3: A color compensation control tubes were prepared similarly for all assay types. 20 L of prepared RBC were pipetted into two separate tubes. To one tube 10 μl of first fluorochrome conjugated antibody reagent i.e., panHb-FITC (Tube 2) was added, and into another tube 10 μl of the second fluorochrome conjugated antibody reagent i.e. Glycophorin A-PE (Tube 3) was added. Each tube was vortexed for 5 sec. and incubated at room temperature for 10-15 min. Each tube was washed in a centrifuge with 3 ml of PBS three times and the contents of each tube were resuspended to 0.5 ml with PBS. The contents of both tubes were pooled together to yield 1.0 ml for the color compensation sample tube. FIG. 2A depicts dual color dot plot for color compensation showing the distribution of red cells stained with 1.1 μg Pan-Hb-FITC monoclonal antibody (FL1) (Tube 2) and 0.1 μg Glycophorin-A-PE antibody (FL2) (Tube 3) from the same specimen stained separately and pooled. FIG. 2B depicts the specific staining for Pan-Hb-FITC (Tube 2) with the second peak of the histogram. FIG. 2C depicts specific staining for Glycophorin-A-PE (Tube 3) with the second peak of the histogram. In FIG. 2B and 2C, linear analysis regions are assigned in the histograms for both the negative (first) and positive (second) peaks.

### Example 3

### RBC Antibody Staining

Staining with the monoclonal preparations was conducted by incubating 20 μl of RBC prepared in accordance with Example 1 with the antibody reagent preparation shown in Example 3A-D. Cells were vortexed for 3 seconds and incubated at room temperature for 10 min. Cells were washed twice by centrifugation and resuspended in 1 ml PBS.

### Example 3A

### Detection of HbA_{1C} RBC (HbA_{1C}/Pan Hb)

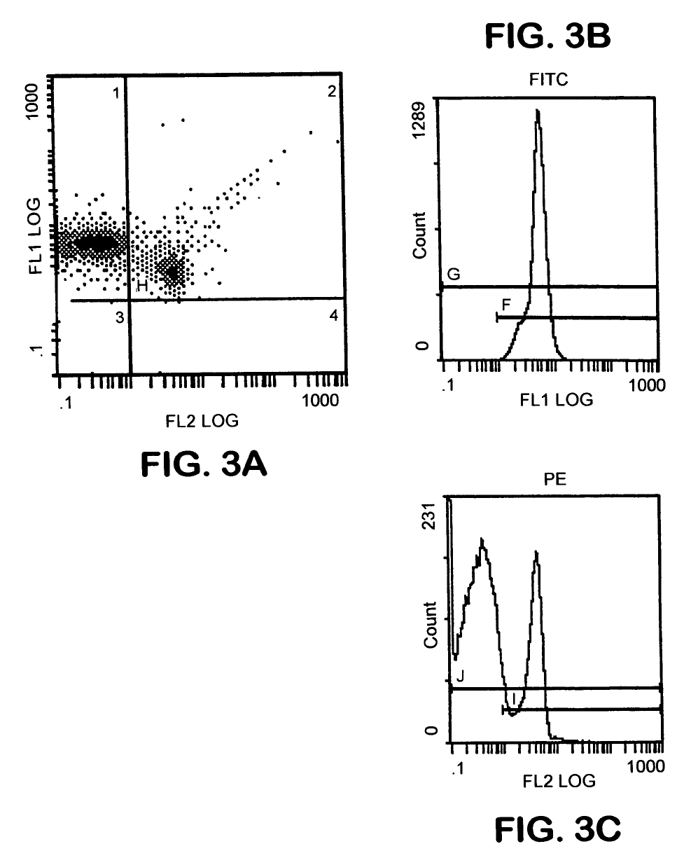

RBC from whole blood were prepared in accordance with Example 1 and were stained with 10 μl (1.1 μg) of PanHb-PE and 10 μl (2.0 μg) of HbA_{1C}-FITC. FIG. 3A is a dot plot with the relative distribution (%) of red cells containing Hb A_{1C} and Pan-Hb (Quadrant 2) and only Pan Hb (Quadrant 1). FIG. 3B depicts the specific staining for Pan-Hb-FITC with the right peak of the histogram. FIG. 3C depicts the specific staining for Hb A_{1C} with the right peak of the histogram. In FIG. 3B and 3C, linear analysis regions are assigned in the histograms for both the negative (first) and positive (second) peaks.

### Example 3B

### Detection of HbS RBC (Hb S/Pan Hb)

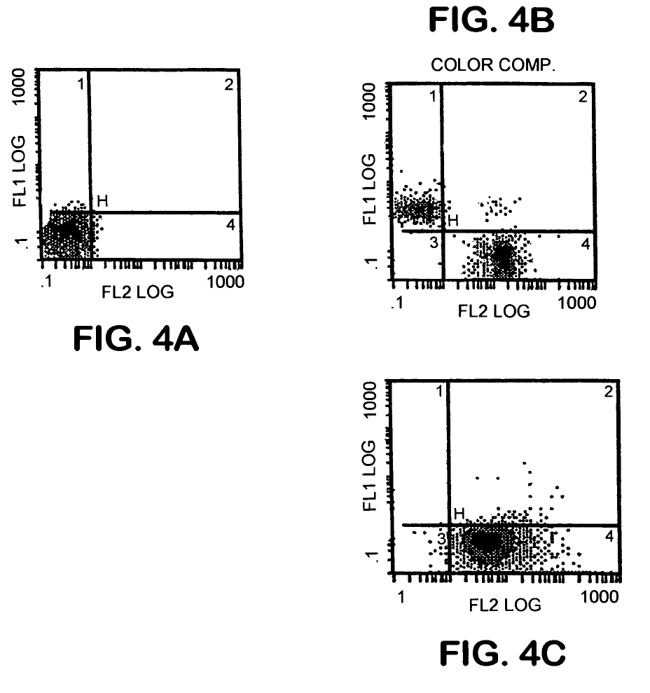

An Immuno-Trol™ control product sample preparation from normal whole blood “contaminated” with a known number of RBCs derived from a sickle cell patient was stained as above using 10 μl (1.1 μg) of panHb-PE and 30 μl (2 μg) of HbS-FITC. FIG. 4A depicts a dual color dot plot for isotype control using MslgG-FITC/MslgG-PE for background voltage setting for Immuno-Trol™ cells. FIG. 4B depicts dual color dot plot for color compensation showing the distribution of red cells stained with 0.1 μg glycophorin A-FITC monoclonal antibody (FL1) and 1.1 μg Pan-Hb-PE monoclonal antibody (FL2). FIG. 4C depicts the dot plot with relative distribution (%) of red cells stained with 2 μg anti Hb S-FITC and 1.1 μg Pan Hb-PE fluorescent reagents. Cells in Quadrant 2 contain Hb S and Pan Hb while cells in Quadrant 4 contain only Pan Hb.

### Example 3C

### Detection of i Antigen in Immuno-Trol™ Control Product and Blood RBC's (i-Antigen/HbF)

i antigen in RBCs was assayed by detecting Hb F in cell preparations containing known number of cord blood RBCs. Two types of preparations were generated by spiking Immuno-Trol™ control product and peripheral blood from a health donor with cord blood containing 1.5% and 0.5% of i antigen-containing cells respectively. Staining was done as above.

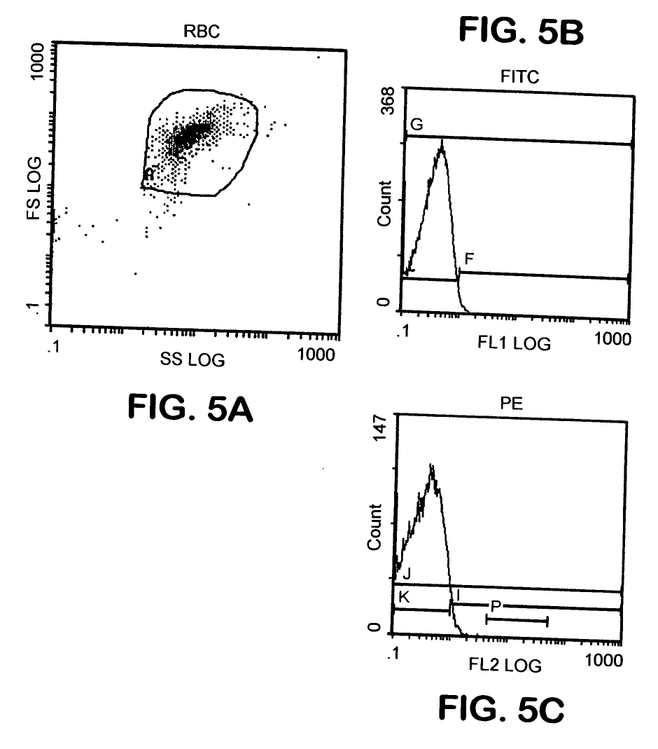

FIG. 5A-C comprises one scatterplot and two histograms which relate to the control product. FIG. 5A depicts a representative dot plot showing Immuno-Trol™ cells distribution gated on forward versus side angle light scattering (log scale). FIG. 5B depicts the background fluorescence for FL1 as determined by an MslgG1-FITC isotype control (log scale). FIG. 5C depicts the background fluorescent for FL2 as determined by an MslgG1-PE isotype control (log scale). Linear analysis regions are assigned in both histograms of FIGS. 5B and 5C.

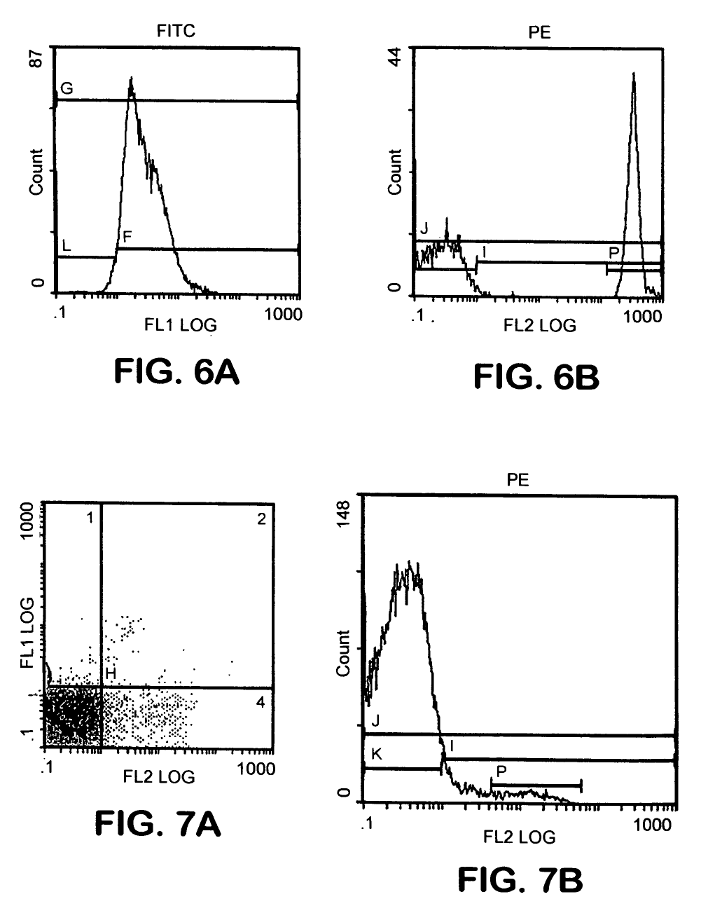

FIG. 6A-B comprises two histograms which relate to color compensation using single color histograms for color compensation showing the distribution of Immuno-Trol™ cells stained with 1.1 μg Pan-Hb-FITC monoclonal antibody (FL1) and 0.1 μg Glycophorin A-PE monoclonal antibody (FL2) in FIG. 6A and FIG. 6B respectively. Linear analysis regions are assigned in both histograms of FIGS. 6A and 6B.

FIG. 7A-B comprises one scatterplot and one histogram that relate to a test assay of hemoglobins. FIG. 7A depicts a dot plot with the relative distribution (%) of containing i antigen (FL2) and HbF (FL1) in Quadrant 2 and only HbF in Quadrant 1. FIG. 7B depicts the specific staining for i antigen, second peak. Linear analysis regions were assigned in the histogram of FIG. 7B.

### Example 3D

### Test Sample Analysis

RBC from whole blood were prepared in accordance with Example 1 and spiked with cord blood. A test sample of blood was reacted with pan hemoglobin antibody conjugate and anti i antigen antibody.

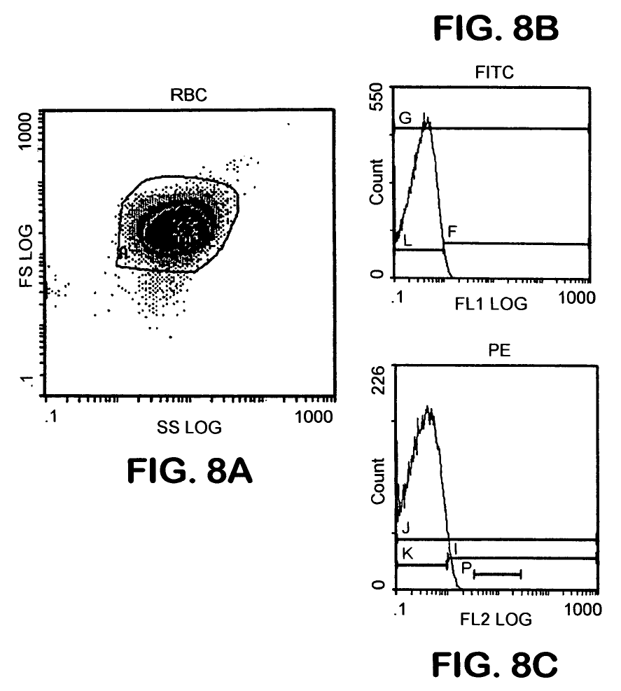

FIG. 8A-C comprises one scatterplot and two histograms that relate to the control product. FIG. 8A depicts a representative dot plot indicating the distribution of RBC preparation gated on forward versus side angle light scattering (log scale). FIG. 8B depicts the background fluorescent for FL1 as determined by MslgG1-FITC isotype control (log scale). FIG. 8C depicts the background fluorescent for FL2 as determined by MslgG1-PE isotype control (log scale). Linear analysis regions were assigned in the histograms of FIGS. 8B and 8C.

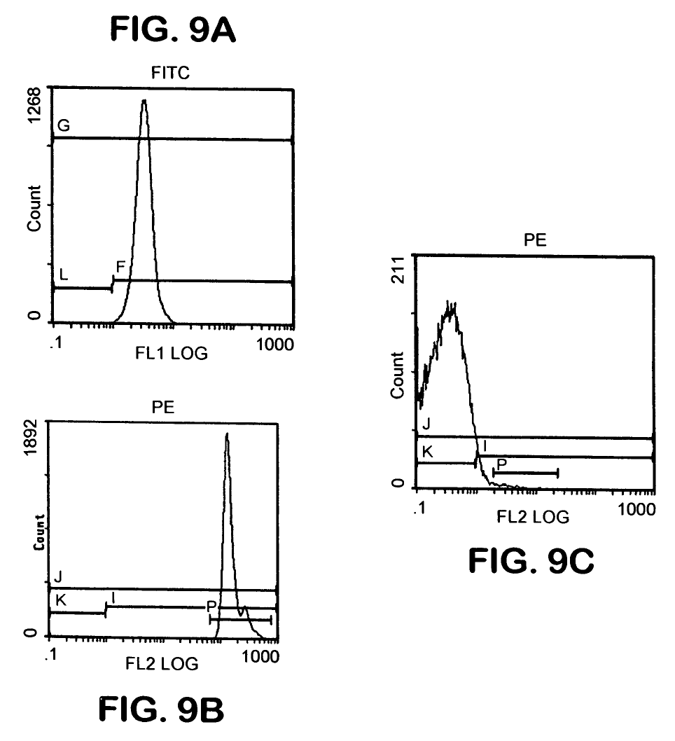

FIG. 9A-C comprises three histograms that relate color compensation and test assay. FIG. 9A depicts a histogram indicating the distribution of normal red cells spiked with cord blood red blood cells and stained for 1.1 μg Pan-Hb-FITC monoclonal antibody (FL1). FIG. 9B depicts a histogram showing the distribution of the red cells stained with and 0.1 μg Glycophorin A-PE monoclonal antibody (FL2). FIG. 9C depicts a histogram showing the red cells stained with i antigen. The small positive peak (P cursor) indicates cells specifically stained for i antigen. Linear analysis regions were assigned for all histograms.

### Example 4

### Analysis on Flow Cytometer

While the presented studies were conducted using a single laser Beckman Coulter XL™ flow cytometer, they can also be performed using other flow cytometers. After running appropriate quality control products to ensure proper instrument performance, Tube 1, prepared according to Example 2, was analyzed for background fluorescence and non-specific binding to set voltages of the flow cytometer. Combined tubes 2 and 3, also prepared according to Example 2, were used as a color compensation control. Tube 4 was prepared using RBC prepared according to Example 1 and stained with an antibody conjugate according to the general procedure provided in Example 3. Tube 4 was used as a dual color sample to determine percent positive of RBC with Hb of interest. The results of flow cytometry analysis of the samples are shown in FIGS. 8A-9C which are scatter plots and histograms of a dual color flow cytometry analysis of a normal blood sample spiked with cord blood red blood cells labeled with pan hemoglobin antibody and anti i antigen antibody.

FIG. 8A-C comprises one scatterplot and two histograms that relate to control products. FIG. 8A depicts a representative dot plot indicating the distribution of RBC preparation gated on forward versus side angle light scattering (log scale). FIG. 8B depicts the background fluorescent for FL1 as determined by MslgG1-FITC isotype control (log scale). FIG. 8C depicts the background fluorescent for FL2 as determined by MslgG1-PE isotype control (log scale). Linear analysis regions were assigned in the histograms of FIGS. 8B and 8C.

FIG. 9A-C comprises three histograms that relate color compensation and test assay. FIG. 9A depicts a histogram indicating the distribution of normal red cells spiked with cord blood red blood cells and stained for 1.1 μg Pan-Hb-FITC monoclonal antibody (FL1). FIG. 9B depicts a histogram showing the distribution of the red cells stained with and 0.1 μg Glycophorin A-PE monoclonal antibody (FL2). FIG. 9C depicts a histogram showing the red cells stained with i antigen. The small positive peak (P cursor) indicates cells specifically stained for i antigen. Linear analysis regions were assigned for all histograms.

The specification is understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

1. A method of analyzing a hemoglobin type or variant in a test sample, which comprises:
a) mixing a test sample from a patient with a pan-hemoglobin antibody that is conjugated to a first label and a hemoglobin type or variant-specific affinity reagent that is conjugated to a second label; and
b) measuring the test sample to determine a signal generated from the first label on the pan-hemoglobin antibody and a signal generated from the second label on the hemoglobin type or variant-specific affinity reagent; and
c) comparing the signal from said pan-hemoglobin antibody and said hemoglobin type or variant specific affinity reagent.

2. The method of claim 1, wherein said comparing of the signal from said pan-hemoglobin antibody and from said hemoglobin type or variant specific affinity reagent comprises a determination of the percentage of red blood cells that contain the type or variant of hemoglobin.

3. The method of claim 1, wherein said comparing of the signal from said pan-hemoglobin antibody and from said hemoglobin type or variant specific affinity reagent comprises a determination of the percent concentration of the hemoglobin type or variant.

4. The method of claim 1, wherein said comparing of the signal from said pan-hemoglobin antibody and said hemoglobin type or variant specific affinity reagent comprises a determination of the mean number of red blood cells per blood volume containing the hemoglobin type or variant.

5. The method of claim 1, which further comprises mixing the test sample with an additional hemoglobin type or variant-specific affinity reagent that is conjugated to a third label and measuring the signal generated from the third label of the hemoglobin type or variant antibody.

6. The method of claim 1, which further comprises comparing the signal of said pan-hemoglobin antibody and said hemoglobin type or variant specific affinity reagent to a reference value.

7. The method of claim 6, wherein the reference value comprises a value from comparing a signal from an pan-hemoglobin antibody and from a hemoglobin type or variant specific affinity reagent in a normal patient population.

8. The method of claim 6, wherein the reference value comprises a previous comparison of the signal from said pan-hemoglobin antibody and said hemoglobin type or variant specific affinity reagent from the same patient.

9. The method of claim 1, wherein the hemoglobin type or variant comprises hemoglobin A_{1C}.

10. The method of claim 6, wherein comparing the signal of said pan-hemoglobin antibody and said hemoglobin type or variant specific affinity reagent to a reference value enables the analysis of a patient condition for diabetes mellitus.

11. The method of claim 6, wherein comparing the signal of said pan-hemoglobin antibody and said hemoglobin type or variant specific affinity reagent to a reference value enables the analysis of a patient condition for hemoglobinopathy.

12. The method of claim 1, wherein the hemoglobin type or variant-specific affinity reagent comprises an antibody conjugated to a fluorochrome.

13. A conjugated antibody product comprising a pan-hemoglobin antibody conjugated to a detectable label.

14. The conjugated antibody product of claim 13, wherein the detectable label is a fluorophore.

15. The conjugated antibody product of claim 13, wherein the antibody binds to a common antigenic determinant on hemoglobin chains.

16. The conjugated antibody product of claim 13, which further comprises at least one additional hemoglobin type or variant-specific affinity reagent that is conjugated to a detectable label and wherein each detectable label is different.

17. The conjugated antibody product of claim 13, which further comprises an erythrocyte specific affinity reagent that is conjugated to a detectable label and wherein each detectable label is different.

18. The conjugated antibody product of claim 13, which further comprises at a leukocyte specific affinity reagent that is conjugated to a detectable label and wherein each detectable label is different.

19. The conjugated antibody product of claim 13, wherein the product comprises a lyophilized product.

20. The conjugated antibody product of claim 13, wherein the product comprises a liquid product containing at least one preservative.

21. The conjugated antibody product of claim 13, which further comprises a known quantity of at least one hemoglobin type or variant.
